(19) United States
(12) Patent Application Publication
(10) Pub. No.: US 2026/0176772 A1
(43) Pub. Date: Jun. 25, 2026

(54) HYDROGEN GENERATOR WITH PRESSURE RELIEF FUNCTION

(71) Applicant: Hsin-Yung LIN, Taoyuan City (TW)

(72) Inventor: Hsin-Yung LIN, Taoyuan City (TW)

(21) Appl. No.: 18/719,638

(22) PCT Filed: Nov. 9, 2022
(86) PCT No.: PCT/CN2022/130795

§ 371 Date: Jun. 13, 2024

(30) Foreign Application Priority Data
Dec. 16, 2021 (CN) 202111541401.4

(51) Int. Cl. C25B 1/04; C25B 9/73

(52) U.S. Cl. CPC C25B 1/04; C25B 9/73; C01B 2203/0233

(57) ABSTRACT

A hydrogen generator with pressure relief function includes a water tank having an accommodation space for accommodating electrolytic water, an electrolysis module arranged in the accommodation space of the water tank to electrolyze the electrolytic water from the water tank to generate gas comprising hydrogen, a humidifying cup arranged above the water tank to humidify the gas comprising hydrogen and having a humidifying chamber and a gas flow channel which are isolated from each other, and the gas flow channel is connected with the water tank. A first valve component is configured to selectively connect the humidifying chamber and an external environment, and a second valve component is configured to selectively connect the accommodation space and the humidifying chamber. When the electrolysis module stops operating, an external air from the external environment enters into the accommodation space through the first valve component and the second valve component.

### BACKGROUND OF THE INVENTION

### 1. Field of the Invention

The present invention relates to a hydrogen generator, and more particularly, to a hydrogen generator with the function of balancing the pressure of a water tank.

### 2. Description of the Prior Art

People always pay much attention to life extension, so many medical technologies are developed to fight diseases. Most medical treatments in the past used passive treatments, which means providing medical treatments when diseases occurred, such as surgeries, medication administration, chemotherapy, radiation therapies for cancer, or management, rehabilitation, and correction of chronic conditions. However, the development of medical technology has been concentrating on preventive medical methods, such as research of healthy food, genetic disease screening and disease prevention, and even has been actively taking initiatives to prevent possible future diseases.

In addition, in order to extend life expectancy of human beings, numerous anti-aging and anti-oxidant technologies, including skincare products and anti-oxidant foods/drugs, have been gradually developed and widely adopted by the public. Researches show that the body's unstable oxygen gas (O+), also known as free radicals (harmful free radicals), caused by various reasons (such as disease, diet, environment or living habits), can mix with inhaled hydrogen gas to form some of the water and be excreted by the body. Namely, by indirectly reducing the number of free radicals in the human body, the reduction of acidic physique is achieved to become healthy alkaline physique, which can resist oxidation and anti-aging, as well as achieve the effects of eliminating chronic diseases and beauty care. Moreover, by increasing the amount of hydrogen gas inhaled, the increase of the time spent on inhaling hydrogen gas (for example, inhaling hydrogen gas during sleeping time) may also efficiently improve the effect of hydrogen gas inhalation.

Currently, hydrogen generators on the market typically generate hydrogen through electrolytic water. When the hydrogen generator is operated to generate hydrogen, the temperature of the water tank increases due to electrolysis by the electrolysis module. On the other hand, when the electrolysis module stops operating, the working temperature of the electrolysis module gradually decreases, resulting in a decrease in gas pressure inside the water tank, which creates negative pressure relative to the outside. Since the pipelines in hydrogen generator of the prior art are closed, the pressure inside the water tank cannot be balanced with the pressure of the external environment, which will cause the water tank to deform. In addition, other components of the hydrogen generator may also include other liquids. These liquids may flow back into the water tank due to the negative pressure in the water tank, thereby contaminating the electrolytic water to reduce the electrolysis efficiency. Furthermore, if too many of these liquids flow back to the water tank, and then cause the water level in the tank to become too high, it will also lead to rupture of the water tank.

Therefore, it is necessary to develop a new type of hydrogen generator to solve the problems of the prior art.

### SUMMARY OF THE INVENTION

Therefore, the present invention provides a hydrogen generator with pressure relief function, which has a simple structure, convenient operation and maintenance, to solve the problems of the prior art, so as to improve safety, electrolysis efficiency, and reducing costs.

In one embodiment of the present invention, the hydrogen generator with pressure relief function comprises a water tank, an electrolysis module, a humidifying cup, a first valve component, and a second valve component. The water tank has an accommodation space for accommodating an electrolytic water. The electrolysis module is configured in the accommodation space of the water tank. The electrolysis module is configured to receive and electrolyze the electrolytic water from the water tank to generate and output a gas comprising hydrogen. The humidifying cup is configured above the water tank. The humidifying cup has a humidifying chamber and a gas flow channel. The humidifying cup is configured to receive and humidify the gas comprising hydrogen. The humidifying chamber and the gas flow channel are isolated from each other, and the gas flow channel is connected with the water tank. The first valve component is configured to selectively connect the humidifying chamber and an external environment. The second valve component is configured to selectively connect the accommodation space and the humidifying chamber. Wherein, when the electrolysis module stops operating, an external air from the external environment enters into the accommodation space through the first valve component and the second valve component.

Wherein, when the electrolysis module operates, the first valve component and the second valve component are closed. When the electrolysis module stops operating, the first valve component and the second valve component are opened, and the accommodation space, the gas flow channel, the first valve component and the second valve component form a continuous channel for allowing the external air to enter into the water tank to balance the internal pressure of the water tank.

Wherein, the hydrogen generator with pressure relief function further comprises an integrated channel device arranged above the water tank. The integrated channel device comprises a gas input channel for receiving the gas comprising hydrogen and a gas output channel for outputting the gas comprising hydrogen. Wherein, the first valve component and the second valve component are arranged in the integrated channel device.

Wherein, the hydrogen generator with pressure relief function further comprises a nebulizer coupled to the gas output channel for receiving the gas comprising hydrogen. Wherein, the nebulizer selectively generates an atomized gas to be mixed with the gas comprising hydrogen to form a healthy gas.

Wherein, the hydrogen generator with pressure relief function further comprises a condensation filter device coupled to the integrated channel device. Wherein, the humidifying cup comprises a connecting chamber for communicating the water tank and the integrated channel device, and the condensation filter device is configured to receive and filter the gas comprising hydrogen from the connecting chamber.

Wherein, the hydrogen generator with pressure relief function further comprises a refining device configured in the humidifying chamber of the humidifying cup and coupled with the condensation filter device. The refining device is configured to refine the gas comprising hydrogen filtered by the condensation filter device, so as to evenly distribute the gas comprising hydrogen in the humidifying chamber.

Wherein, the hydrogen generator with pressure relief function further comprises a hydrogen water cup for accommodating a liquid. Wherein, the hydrogen water cup is configured to input the gas comprising hydrogen into the liquid to generate a liquid comprising hydrogen.

Wherein, the hydrogen generator with pressure relief function further comprises an ozone generator. The ozone generator is coupled to a gas flow channel which is formed between the integrated channel device and the electrolysis module for the gas comprising hydrogen to flow therein. Wherein the ozone generator is configured to generate an ozone gas into the gas flow channel to sterilize the gas flow channel when the electrolysis module stops electrolyzing.

Wherein, the hydrogen generator with pressure relief function further comprises a germ filtering device configured at an air outlet of the hydrogen generator for filtering out germs from the gas comprising hydrogen at the air outlet.

Wherein, the pH value of the electrolytic water in the electrolysis module is in a range between 13˜13.9.

In another embodiment of the present invention, the hydrogen generator with pressure relief function comprises a water tank, an electrolysis module, an integrated channel device, a pressure relief module, and a humidifying cup. The water tank has an accommodation space for accommodating an electrolytic water. The electrolysis module is configured in the accommodation space of the water tank. The electrolysis module is configured to receive and electrolyze the electrolytic water from the water tank to generate and output a gas comprising hydrogen. The integrated channel device is arranged above the water tank. The integrated channel device comprises a gas input channel for receiving the gas comprising hydrogen and a gas output channel for outputting the gas comprising hydrogen. The pressure relief module is configured to operate to make an external air from the external environment enter into the water tank when the electrolysis module stops operating. The humidifying cup is coupled with the integrated channel device for receiving and humidifying the gas comprising hydrogen. Wherein, the pH value of the electrolytic water in the electrolysis module is in a range between 12˜14.

Wherein, the hydrogen generator with pressure relief function further comprises a nebulizer coupled to the gas output channel for receiving the gas comprising hydrogen. Wherein, the nebulizer selectively generates an atomized gas to be mixed with the gas comprising hydrogen to form a healthy gas.

Wherein, the hydrogen generator with pressure relief function further comprises a germ filtering device arranged at an air outlet of the hydrogen generator for filtering out germs from the gas comprising hydrogen at the air outlet.

Wherein, the hydrogen generator with pressure relief function further comprises an ultraviolet light generator arranged in the hydrogen generator for generating an ultraviolet light to sterilize the hydrogen generator.

Wherein, the hydrogen generator with pressure relief function further comprises a condensation filter device coupled to the integrated channel device. Wherein the humidifying cup comprises a connecting chamber for communicating the water tank and the integrated channel device, and the condensation filter device is configured to receive and filter the gas comprising hydrogen from the connecting chamber.

Wherein, the hydrogen generator with pressure relief function further comprises a hydrogen water cup for accommodating a liquid. Wherein, the hydrogen water cup is configured to input the gas comprising hydrogen into the liquid to generate a liquid comprising hydrogen.

Wherein, the hydrogen generator with pressure relief function further comprises an ozone generator. The ozone generator is coupled with a gas flow channel which is formed among the integrated channel device, the electrolysis module and the hydrogen water cup for the gas comprising hydrogen to flow therein. Wherein, the ozone generator is configured to generate an ozone gas into the gas flow channel to sterilize the gas flow channel when the electrolysis module stops electrolyzing.

Wherein, the pressure relief module further comprises a first valve component and a second valve element. The first valve component is configured to selectively connect with the humidifying chamber and the external environment. The second valve element is configured to selectively connect with the accommodation space and the humidifying chamber. When the electrolysis module stops operating, the first valve component and the second valve element are opened, an external air from the external environment enters into the accommodation space through the first valve component and the second valve component.

Wherein, the hydrogen generator with pressure relief function further comprises a flame arrester coupled with the first valve component.

Wherein, the hydrogen generator with pressure relief function further comprises a case and a bracket. The case is configured to accommodate the water tank, the electrolysis module, the integrated channel device, the condensation filter device, the pressure relief module and the humidifying cup. The bracket is configured to support a water bucket. The bracket is arranged on the outside of the case and the water bucket is configured to provide a supplementary water for flowing through the condensation filter device into the water tank. Wherein, the hydrogen generator with pressure relief function further comprises a gas baffle device arranged in the connecting chamber for reducing or avoiding moisture from the gas comprising hydrogen and electrolyte flowing into the integrated channel device.

In summary, the hydrogen generator with pressure relief function of the present invention can balance the pressure inside the water tank by controlling the first valve component and the second valve component to be opened to introduce external air to flow therein after the electrolysis module stops operating, thereby avoiding deformation of the water tank due to pressure differentials and enhancing safety. Moreover, the hydrogen generator with pressure relief function of the present invention can avoid liquids flowing back to the water tank by controlling the first valve component and the second valve component to be opened to block liquids, so as to prevent contamination in the electrolytic water in the water tank or the risk of the water tank bursting due to excessive water levels, thereby improving electrolysis efficiency and safety. Furthermore, due to the alkaline electrolysis environment and the temperature conditions of the hydrogen generator with pressure relief function of the present invention, the environment is unfavorable for bacteria survival, and due to the different devices such as filtering devices, ultraviolet light devices and ozone generators can continuously undergo self-disinfection and self-sterilization to not only provide users with bacteria-free, pure gas comprising hydrogen or health caring gas but also significantly reducing the maintenance costs of the hydrogen generator. Additionally, the hydrogen generator with pressure relief function of the present invention can maintain electrolysis efficiency by backwashing electrolytes with an external water bucket and maintaining the electrolyte concentration in the electrolytic water through the gas baffle assembly to prevent electrolytes from flowing into the humidifying chamber. Moreover, by installing a flame arrester on the continuous channel, the hydrogen generator with pressure relief function of the present invention can prevent the spread of accidentally ignited gas to the outside of the hydrogen generator, thereby enhancing safety.

### BRIEF DESCRIPTION OF THE APPENDED DRAWINGS

FIG. 1 is a structural schematic diagram illustrating the hydrogen generator with pressure relief function according to an embodiment of the present invention.

FIG. 2 is a functional block diagram illustrating the hydrogen generator with pressure relief function of FIG. 1.

FIG. 3 is an exploded diagram illustrating the hydrogen generator with pressure relief function of FIG. 1.

FIG. 4 is an exploded diagram illustrating the water tank of FIG. 1.

FIG. 5A is a structural schematic diagram illustrating the hydrogen generator with pressure relief function of FIG. 1 in another one perspective.

FIG. 5B is a structural schematic diagram illustrating the hydrogen generator with pressure relief function of FIG. 5A, excluding the condensation filter device.

FIG. 5C is a sectional diagram illustrating the sectional view along line A-A of FIG. 5A.

FIG. 6A is a sectional diagram illustrating the sectional view along line B-B of FIG. 5B.

FIG. 6B is a sectional diagram illustrating the sectional view along line C-C of FIG. 5B.

FIG. 7 is a structural schematic diagram illustrating a hydrogen generator with pressure relief function according to another embodiment of the present invention.

FIG. 8 is an exploded diagram illustrating the integrated channel device of FIG. 7.

FIG. 9 is a schematic diagram illustrating a hydrogen generator with pressure relief function according to another embodiment of the present invention.

### DETAILED DESCRIPTION OF THE INVENTION

For the sake of the advantages, spirits and features of the present invention can be understood more easily and clearly, the detailed descriptions and discussions will be made later by way of the embodiments and with reference of the diagrams. It is worth noting that these embodiments are merely representative embodiments of the present invention, wherein the specific methods, devices, conditions, materials and the like are not limited to the embodiments of the present invention or corresponding embodiments. Moreover, the devices in the figures are only used to express their corresponding positions and are not drawing according to their actual proportion.

In the description of the present invention, it is to be understood that the orientations or positional relationships of the terms “longitudinal, lateral, upper, lower, front, rear, left, right, top, bottom, inner, outer” and the like are based on the orientation or positional relationship shown in the drawings. It is merely for the convenience of the description of the present invention and the description of the present invention, and is not intended to indicate or imply that the device or component referred to has a specific orientation, is constructed and operated in a specific orientation, and therefore cannot be understood as limitations of the invention.

In the description of this specification, the description with reference to the terms “a specific embodiment”, “another specific embodiment” or “parts of specific embodiments” etc. means that the specific feature, structure, material or feature described in conjunction with the embodiment include in at least one embodiment of the present invention. In this specification, the schematic representations of the above-mentioned terms do not necessarily refer to the same embodiment. Moreover, the described specific features, structures, materials or characteristics can be combined in any one or more embodiments in a suitable manner.

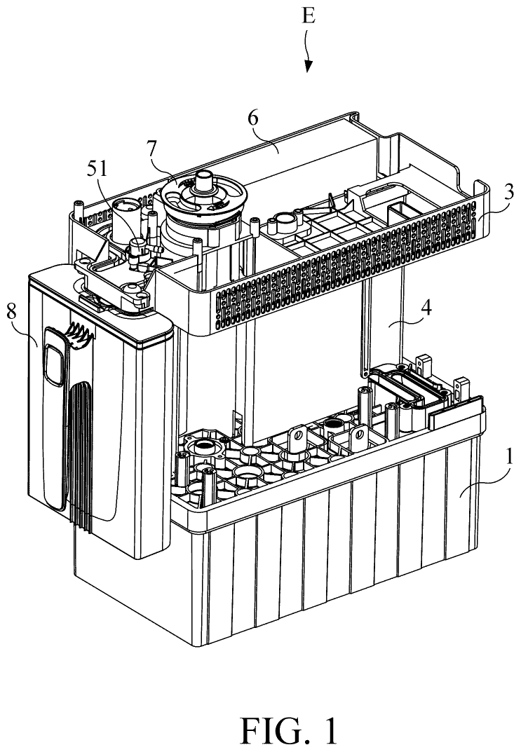

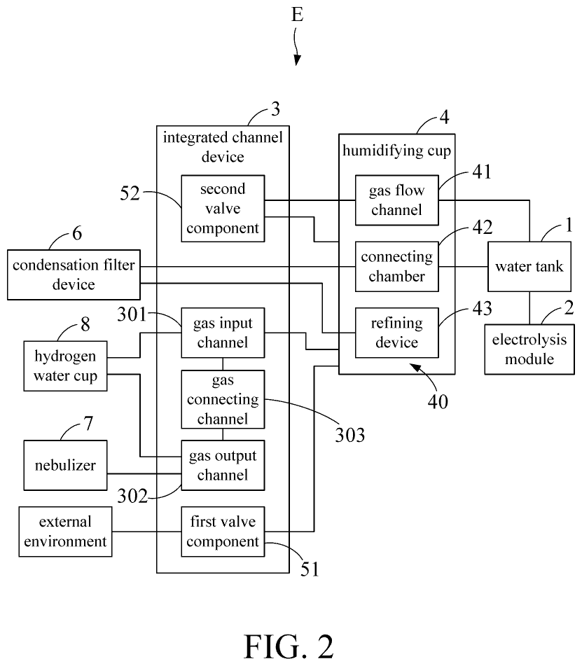

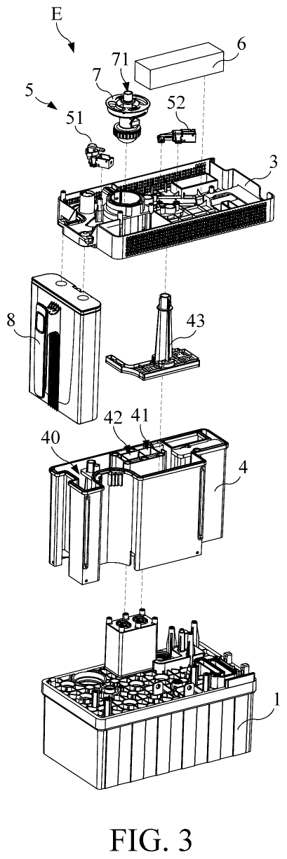

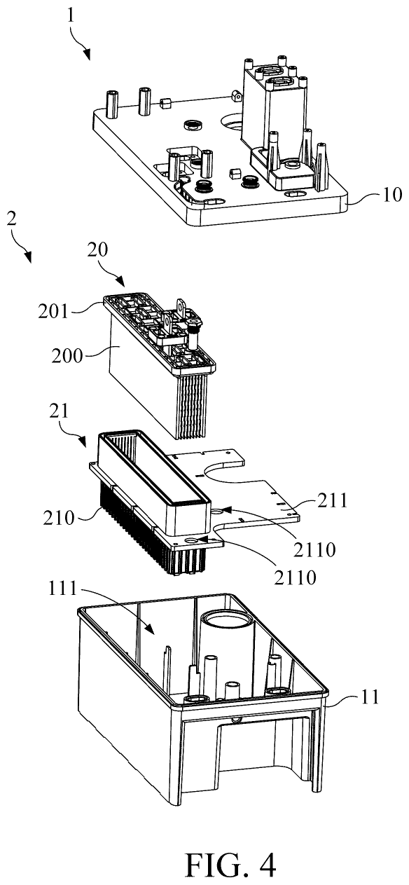

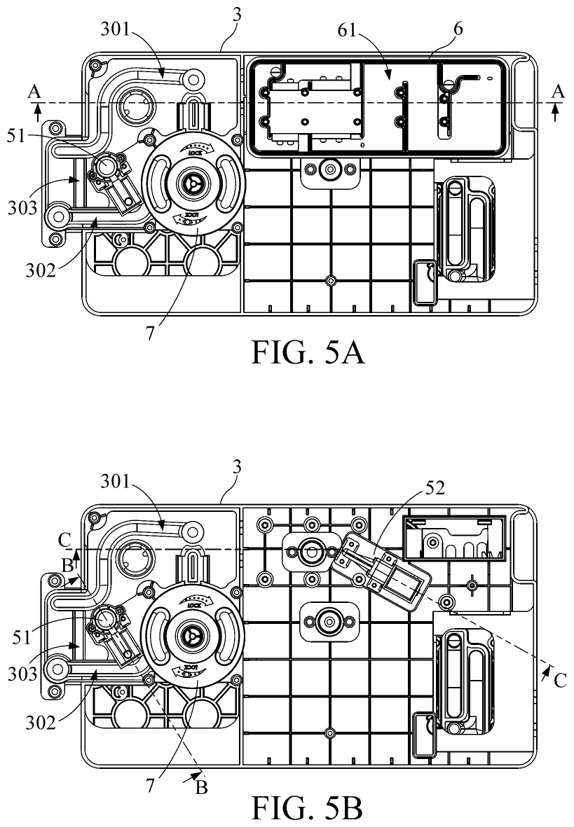

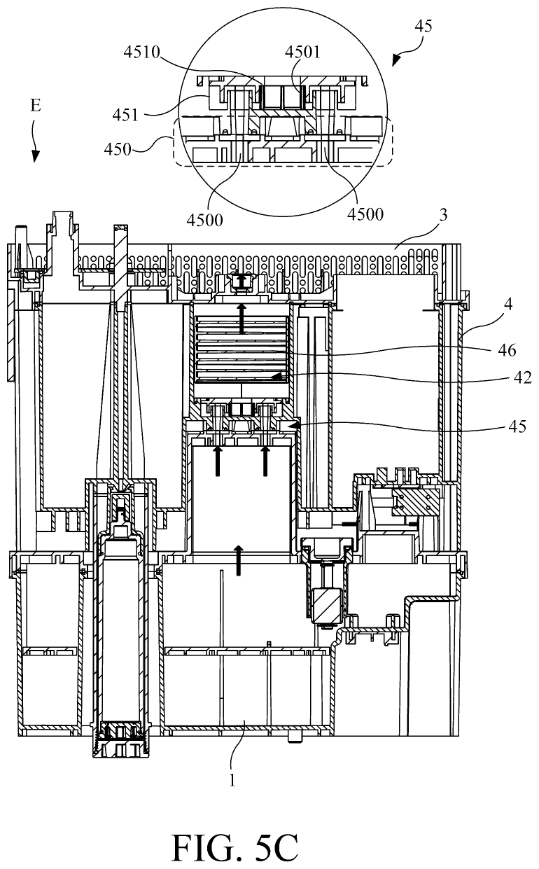

Please refer to FIG. 1 to FIG. 5A. FIG. 1 is a structural schematic diagram illustrating the hydrogen generator E with pressure relief function according to an embodiment of the present invention. FIG. 2 is a functional block diagram illustrating the hydrogen generator E with pressure relief function of FIG. 1. FIG. 3 is an exploded diagram illustrating the hydrogen generator E with pressure relief function of FIG. 1. FIG. 4 is an exploded diagram illustrating the water tank 1 of FIG. 1. FIG. 5A is a structural schematic diagram illustrating the hydrogen generator E with pressure relief function of FIG. 1 in another one perspective. As shown in FIG. 1 to FIG. 5A, in the present embodiment, the hydrogen generator E with pressure relief function includes a water tank 1, an electrolysis module 2, a humidifying cup 4, and a pressure relief module 5. Wherein, the pressure relief module 5 includes a first valve component 51, and a second valve component 52. The water tank 1 has an accommodation space 111 for accommodating an electrolytic water. The electrolysis module 2 is arranged in the accommodation space 111 of the water tank 1, and the electrolysis module 2 is configured to receive and electrolyze the electrolytic water from the water tank 1 to generate and output a gas comprising hydrogen. The humidifying cup 4 is arranged above the water tank, and is configured to receive and humidify the gas comprising hydrogen. The humidifying cup 4 includes a humidifying chamber 40 and a gas flow channel 41. The humidifying chamber 40 and the gas flow channel 41 are isolated from each other, and the gas flow channel 41 is connected with the water tank 1. The first valve component 51 is coupled with the humidifying chamber 40 and is configured to selectively connect the humidifying chamber 40 and an external environment. The second valve component 52 is coupled with the gas flow channel 41 and is configured to selectively connect the accommodation space and the humidifying chamber.

In the specific embodiment, the water tank 1 includes a lid 10 and a tank body 11, wherein the tank body 11 forms an accommodation space 111 for accommodating electrolytic water, and the lid 10 can be covered on the tank body 11. The electrolysis module 2 can be installed in the accommodation space 111 of the water tank 1 and be used to electrolyze the electrolytic water to generate and output the gas comprising hydrogen. The electrolysis module 2 includes an electrode plate assembly 20, and an electrolysis tank fixing plate 21. The electrolysis tank fixing plate 21 includes an electrolysis tank body 210 and a partition plate 211. The electrode plate assembly 20 can be accommodated in the electrolysis tank body 210 of the electrolysis tank fixing plate 21. The electrode plate assembly 20 includes a plurality of electrode plates 200 and a spacer plate 201 connecting each electrode plate 200. The spacer plate 201 is arranged on the upper surface of each electrode plate 200 to space multiple electrode plates 200 apart to form multiple electrode flow channels when the electrode plate assembly 20 is accommodated in the electrolysis tank body 210. The partition plate 211 can fix the electrolysis module 2 in the water tank 1 and divide the water tank 1 into upper and lower layers. Wherein, the electrolytic water is located in the lower layer, and the gas comprising hydrogen generated by electrolysis is mainly located in the upper layer. In order to maintain circulation between the upper and lower layers, the partition plate 211 has a plurality of through holes 2110 to communicate with the upper layer and the lower layer. The electrolysis tank fixing plate 21 can be a one-piece formed structure. In addition, it should be understood that those skilled in the art can adjust the shape of the partition plate 211 according to their needs to provide space for the installation of other components.

In the specific embodiment, the pH value of the electrolytic water electrolyzed by the hydrogen generator E of the present invention with pressure relief function is between 11˜14. However, in practice, it is not limited hereto; the electrolytic water can also contain weak alkaline electrolytes to provide the electrolysis module 2 to electrolysis to generate the gas comprising hydrogen. The hydrogen generator E of the present invention with pressure relief function performs electrolysis and generates the gas comprising hydrogen in an alkaline environment, thus the alkaline electrolytic water can sterilize the inside of the hydrogen generator E. In addition, when the electrolysis module 2 is operating, the electrode plate assembly 20 is applied in electrical energy, and then cause multiple electrode plates 200 to form positive and negative electrodes to electrolyze the electrolytic water in the water tank 1. Since part of the electrical energy applied in the electrode plate assembly 20 is converted into heat energy during electrolysis, the operating temperature of the electrolysis module 2 will increases. In this specific embodiment, the operating temperature of the electrolysis module 2 during electrolysis of the electrolytic water is in a range between 55° C. to 85° C. to provide the electrolysis module 2 with sterilizing function as well.

In practice, the electrolytic water in the electrolysis module includes electrolytes with concentrations exceeding 0.1% weight percent or exceeding 0.1% volume percent (for example, the range is between 0.5%˜15%), so as to form a strong alkaline environment with pH value is larger than 12 in the electrolysis module 2 and the water tank 1. Furthermore, if the electrolytic water includes electrolytes with 1% weight percentage concentration or larger than 1% volume percentage concentration or a greater concentration, at this time, the strong alkaline environment in the electrolysis module 2 and the water tank with pH value is larger than 13.4.

It is known from experiments that the electrolytic water in the electrolysis module 2 and the water tank 1 of the present invention includes electrolytes over than 0.1% concentration, at this time, the pH value in the internal environment of the electrolysis module 2 and the water tank can reach above 12. When the electrolytic water includes electrolytes with 1% concentration, the pH value in the electrolytic water can reach to 13.4. When the electrolytic water includes electrolytes with 2% concentration, the pH value in the electrolytic water can reach to 13.7. When the electrolytic water includes electrolytes with 3% concentration, the pH value in the electrolytic water can reach to 13.88. When the electrolytic water includes electrolytes with 4% concentration or over than 4%, the pH value in the electrolytic water can reach to 14. The experimental result shows that there are no germs can survive under the strong alkaline environment with pH value is between 12˜14. However, too much concentration of the electrolytes will easily generate alkali mist, which will easily harm the human respiratory system if the alkali mist will not be filtered. On the contrary, the electrolysis efficiency decreases when the concentration of the electrolytes in the electrolytic water is less than 0.1%. On the other hand, the experimental result shows that when the electrolytic water includes electrolytes with 6% concentration, the hydrogen generator will generate the minimum of the noise. Therefore, according to the above factors such as sterilization, electrolysis efficiency, alkali mist and noise, the suitable electrolyte concentration range of the hydrogen generator of the present invention is between 0.5%˜15% which can form a strong alkaline environment with pH value in the range between 12˜14 in the electrolysis module for effective sterilization. It is known from experiments that if the electrolyte concentration in the electrolyzed water is between 1%˜3%, the above-mentioned strong alkaline environment with pH value is between 13˜13.9.

The experimental result shows that the strong alkaline environment in the water tank 1 and the electrolysis module 2 is enough to prevent all Legionella pneumophila from surviving. In addition, Mycobacterium tuberculosis is a representative of alkali-resistant bacteria. When the electrolytic water includes electrolytes with 0.1% concentration, the pH value in the electrolytic water is larger than 12 to inactivate Mycobacterium tuberculosis. Furthermore, when the electrolytic water includes electrolytes with 1% concentration, the pH value in the electrolytic water is larger than 13.4 or even close to 14, at this time, Mycobacterium tuberculosis or other alkali-resistant bacteria cannot survive.

Next, since the black variant spores of Bacillus subtilis is able to highly resistant to heat, ultraviolet light, ionizing radiation and certain chemicals, the black variant spores of Bacillus subtilis is considered to be the most difficult to be eliminated in bacterial species. Therefore, in some countries such as The United States, the United Kingdom, Japan, the European Union or other countries have added the black variant spores of Bacillus subtilis in food testing and medical testing as a quality control standard testing strain. Moreover, the black variant spores of Bacillus subtilis has been used internationally as an indicator bacterium for evaluation tests of bactericidal effects of chemical disinfectants, dry heat, and ethylene oxide. In addition, the black variant spores of Bacillus subtilis is a representative strain for high-level disinfection methods against disinfectants or disinfection equipment, the Chinese Ministry of Health has also added the black variant spores of Bacillus subtilis as a standard testing strain in the “Technical Specifications for Disinfection”.

The hydrogen generator E with pressure relief function of the present invention also has the ability to eliminate the black variant spores of Bacillus subtilis. As mentioned previously, the pH value in the alkaline environment in the electrolysis module 2 and the water tank 1 of the hydrogen generator is over than 12, and can even reach 13.8 or above. The experimental result shows that the black variant spores of Bacillus subtilis cannot survive in the electrolytic water of the water tank 1 and the electrolysis module 2 which include electrolytes over than 0.1% concentration and the pH value in the alkaline environment is over than 12. In addition, as mentioned above, the gas comprising hydrogen generated by the electrolysis module also contains moisture from the electrolytic water with a high pH value, at this time, the gas comprising hydrogen can also sterilize or disinfect the gas flow channel.

In the specific embodiment, the hydrogen generator E with pressure relief function further includes an integrated channel device 3 and the condensation filter device 6. The humidifying cup 4 is vertically stacked above the water tank 1, the integrated channel device 3 is vertically stacked above the humidifying cup 4, and the condensation filter device 6 is then arranged within the accommodation space of the integrated channel device 3. In practice, the hydrogen generator E with pressure relief function can further include a case for accommodating the aforementioned components.

The humidifying cup 4 includes a humidifying chamber 40, a gas flow channel 41, and a connecting chamber 42. The humidifying chamber 40 contains supplementary water. The gas flow channel 41 and the connecting chamber 42 are isolated from the humidifying chamber 40. The connecting chamber 42 is configured to connect the water tank 1 and the integrated channel device 3 to allow the gas comprising hydrogen generated by the electrolysis module 2 installed in the water tank 1 to pass through the connecting chamber 42 into the integrated channel device 3, and then enter into the condensation filter device 6 through the condensation flow channel 61 of the integrated channel device 3. The condensation filter device 6 has the condensation flow channel 61 which allows the gas comprising hydrogen to flow through to be condensed and be filtered. In this specific embodiment, the condensation filter device 6 can be embedded within the integrated channel device 3 and can be pulled out from the side of the integrated channel device 3 for easy replacement without disassembling the entire hydrogen generator E with pressure relief function. The condensation filter device 6 in FIG. 5A can further include a lift-up structure (the figure is not shown) arranged at the top of the condensation filter device 6 to fix and seal the condensation flow channel 61. The gas comprising hydrogen filtered by the condensation filter device 6 can then flow through the integrated channel device 3 to the humidifying chamber 40 of the humidifying cup 4, where the supplementary water in the humidifying chamber 40 can further filter and humidify the gas comprising hydrogen.

Please refer to FIG. 2, FIG. 3, FIG. 5A, and FIG. 5C. FIG. 5C is a sectional diagram illustrating the sectional view along line A-A of FIG. 5A. The hydrogen generator with pressure relief function E of the present invention further includes a water blocking device 45 and a gas baffle assembly 46, both of which can be installed in the connecting chamber 42. As shown in the partially enlarged schematic diagram of the water blocking device 45 in FIG. 5C, the water blocking device 45 is arranged above the water tank 1 to block the outflow of electrolytic water from the water tank 1 when the water tank 1 tilts at an inclined angle. In practical application, the water blocking device 45 includes a bottom portion 450 (shown in the circled in dashed line in the figure) and a water blocking component 451. The bottom portion 450 has an air inlet 4500 and an elastic pin 4501. The air inlet 4500 is used to receive the gas comprising hydrogen. The water blocking component 451 has a pinhole 4510, and the elastic pin 4501 is resiliently arranged in the pinhole 4510 in a restorable manner, and can press against the water blocking component 451 to keep the air inlet 4500 open. When the water tank 1 or the hydrogen water cup of the hydrogen generator E tilts at an inclined angle, the elastic pin 4501 can compress and slide into the pinhole 4510 to couple the water blocking component 451 with the bottom portion 450, so as to close the air inlet 4500 to block the outflow of the electrolytic water from the water tank 1.

The gas baffle assembly 46 can be arranged above the water blocking device 45. The gas baffle assembly 46 is used to reduce or prevent moisture and electrolytes in the gas comprising hydrogen to flow into the condensation flow channel 61. The gas baffle assembly 46 is composed of multiple interlocking plates. Furthermore, when the gas comprising hydrogen passes through the connecting chamber 42, moisture and electrolytes in the gas comprising hydrogen are condensed on the gas baffle assembly 46, and then be blocked on the gas baffle assembly 46. The condensed moisture and electrolytes on the gas baffle assembly 46 will be vacuum-pumped and drawn back into the water tank 1 through the integrated channel device 3 for flushing, so as to maintain the electrolyte concentration in the electrolytic water also maintain the electrolysis efficiency.

Further, the hydrogen generator with pressure relief function E further includes a refining device 43 arranged in the humidifying chamber 40 of the humidifying cup 4. The refining device 43 connects the humidifying chamber 40 and the condensation flow channel 61 of the condensation filter device 6 to allow the filtered gas comprising hydrogen to be input into the supplementary water in the humidifying chamber 40. Specifically, the surface of the refining device 43 can contain multiple micro bubble structures. The gas comprising hydrogen can pass through the micro bubble structures. When the gas comprising hydrogen enters into the supplementary water through the micro bubble structures, the gas comprising hydrogen will be divided into tiny bubbles. Therefore, the gas comprising hydrogen can be thoroughly filtered and humidified by the supplementary water in the humidifying chamber 40. In summary, when the gas comprising hydrogen generated by the electrolysis module 2 leaves the surface of the water tank 1, the gas comprising hydrogen then enters the connecting chamber 42 of the humidifying cup 4. Next, the gas comprising hydrogen sequentially passes through the connecting chamber 42 of the humidifying cup 4, the condensation flow channel 61 of the condensation filter device 6, the refining device 43, and the humidifying chamber 40 of the humidifying cup 4.

In this specific embodiment, the integrated channel device 3 includes a gas input channel 301 and a gas output channel 302. The hydrogen generator with pressure relief function E further includes a nebulizer 7 coupled to the gas output channel 302 of the integrated channel device 3 to receive the gas comprising hydrogen and optionally generate atomized gas to mix with the gas comprising hydrogen to form health caring gas. The nebulizer 7 can generate the atomized gas, which can be mixed with the gas comprising hydrogen to form health caring gas. Wherein, the atomized gas can be selected from one or a combination of vapor, atomized medication, or volatile essential oils. In one specific embodiment, the nebulizer 7 includes an oscillator. The oscillator atomizes the water, atomized medication, or volatile essential oils by oscillation to generate the atomized gas, then mix the mixed gas and the atomized gas to form the health caring gas. The nebulizer 7 can be selectively opened or closed according to user needs to provide the health caring gas containing mixed atomized gas for inhalation or only provide mixed gas (such as the hydrogen gas diluted with oxygen after the second oxygen dilution) for the user to inhale.

In addition, in this specific embodiment, the hydrogen generator with pressure relief function E further includes a hydrogen water cup 8, and the integrated channel device 3 further includes a gas connecting channel 303. The gas connecting channel 303 can optionally couple to the gas input channel 301 and the gas output channel 302, and the gas input channel 301 and the gas output channel 302 can optionally couple to the hydrogen water cup 8. The hydrogen water cup 8 can be used to accommodate liquid (such as drinking water), and the hydrogen water cup 8 is used to infuse the gas comprising hydrogen to form liquid comprising hydrogen.

Therefore, when the electrolysis module 2 of the hydrogen generator with pressure relief function E electrolyzes the electrolytic water and generates the gas comprising hydrogen, the gas comprising hydrogen flows from the water tank 1 through the connecting chamber 42 of the humidifying cup 4, the condensation flow channel 61 of the condensation filter device 6, the refining device 43, the humidifying chamber 40 of the humidifying cup 4, and the gas input channel 301 of the integrated channel device 3. When the gas comprising hydrogen flows into the gas input channel 301, the gas comprising hydrogen can sequentially flow through the hydrogen water cup 8, the gas output channel 302, and the nebulizer 7, or alternatively, and can sequentially flow through the gas connecting channel 303, the gas output channel 302, and the nebulizer 7 without passing through the hydrogen water cup 8. However, it should be understood that the above-mentioned flow direction of the hydrogen-containing gas is one of the embodiments of the hydrogen gas generator E of the present invention, those skilled in the art can adjust the order of the components according to their needs, and it is not limited to this.

In another one embodiment, the humidifying cup is connected to the gas flow channel which is formed between the water tank, the electrolysis module, the hydrogen water cup, and the condensation filter device and other units through the integrated channel device. The humidifying cup can also transfer the replenishing water through the same path. Namely, the gas flow channel and the liquid flow path use the same path as mentioned, but the flow direction of the gas comprising hydrogen and the replenishing water are opposite. In an experiment, the hydrogen generator E of the present invention is input a large amount of the black variant spores of Bacillus subtilis into the humidifying cup from the outside. The black variant spores of Bacillus subtilis will enter the above-mentioned units, especially the water tank, which can simulate the situation where the electrolytic water in the water tank is contaminated by pathogens. Next, turn on the hydrogen generator and continue operating, measure the microbial load of the gas output from the air outlet of the hydrogen generator.

In the above experiment, after the hydrogen generator was continued to operate for 30 minutes to 1 hour, there is no microorganism detected at the air outlet. Furthermore, after operating for 23.5 hours to 24 hours, there is still no microorganism detected at the air outlet. In addition, after operating for 24 hours, the total number of bacteria in the water tank was reduced by more than 99%. Next, the above experimental results were converted into a log killing value of microorganisms by the electrolysis module, and the value is over than 6.46, which is far exceeding the requirement of a log killing value of 5 for high-level disinfection in the “Technical Specifications for Disinfection”. Through the above experiment, the hydrogen generator of the present invention can eliminate the most difficult-to-eliminate bacteria in the world while maintaining the purity of the output gas, and has excellent disinfection and sterilization effects.

In the above embodiments, although the higher the electrolyte concentration in the electrolytic water, the higher pH value is beneficial to sterilization. However, too high electrolyte concentration may reduce the efficiency of electrolyzing the gas comprising hydrogen. Therefore, in practice, the electrolyte concentration in the electrolytic water can be maintained between 0.5% and 15%, and further between 1% and 3%, which can achieve excellent disinfection and sterilization effects in the alkaline environment with high pH value in the electrolysis module and the water tank, and can make the electrolysis module maintain high production efficiency.

In addition, in the specific embodiment, the gas comprising hydrogen generated from the electrolytic water by the electrolysis module 2 will enter will the gas flow channel and flow through other units or modules in the hydrogen generator E, such as the humidifying cup. It should be noted that the paths through which the gas comprising hydrogen can flow in the hydrogen generator E are all part of the gas flow channel, including the integrated channel device, electrolysis module, and hydrogen water cup as described in the specific embodiments above. The hydrogen generator E can also include filter cotton arranged in the gas flow channel. Therefore, when the gas comprising hydrogen flows through the filter cotton, bacteria inside the hydrogen generator E will be filtered out by the filter cotton.

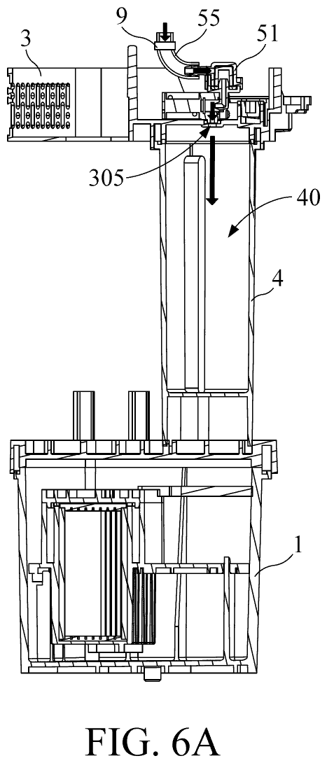

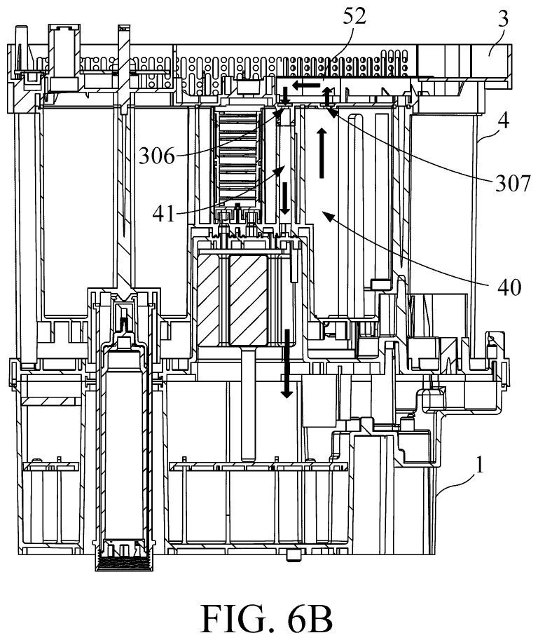

Please refer to FIG. 3, FIG. 5A, FIG. 5B, FIG. 6A, and FIG. 6B. FIG. 5B is a structural schematic diagram illustrating the hydrogen generator E with pressure relief function of FIG. 5A, excluding the condensation filter device. FIG. 6A is a sectional diagram illustrating the sectional view along line B-B of FIG. 5B. FIG. 6B is a sectional diagram illustrating the sectional view along line C-C of FIG. 5B. As shown in FIG. 5, FIG. 6A, and FIG. 6B, the first valve component 51 and the second valve component 52 are arranged on the integrated channel device 3. The first valve component 51 and the second valve component 52 can be two-way solenoid valves, each containing two valve ports. The first valve component 51 and the second valve component 52 can be opened or closed to allow the two valve ports to communicate with each other or to isolate each other. Furthermore, the integrated channel device 3 includes the first opening 305, the second opening 306, and the third opening 307. The first opening 305 communicates with the humidifying chamber 40 and one valve port of the first valve component 51. The second opening 306 and the third opening 307 respectively communicate with the two valve ports of the second valve component 52, and respectively communicate with the gas flow channel 41 and the humidifying chamber 40. In practice, the other valve port of the first valve component 51 can be connected to an air outlet pipe 55, and the air outlet pipe 55 can pass through the case to communicate with the external environment.

In this specific embodiment, when the electrolysis module 2 is operating, the first valve component 51 and the second valve component 52 are closed. At this time, the humidifying chamber 40 is not in communication with the external environment, and the humidifying chamber 40 is not directly connected to the accommodation space 111 of the water tank 1. Therefore, when the electrolysis module 2 electrolyzes the electrolytic water to generate the gas comprising hydrogen, the gas comprising hydrogen sequentially will flow through the connecting chamber 42 of the humidifying cup 4, the condensation filter device 6, and the refining device 43 and then enter into the humidifying chamber 40, the gas comprising hydrogen will not flow through the gas flow channel 41 and the second valve component 52. Additionally, the gas comprising hydrogen or other gases in the humidifying chamber 40 will not flow to the external environment.

When the electrolysis module 2 stops operating, the first valve component 51 and the second valve component 52 are opened. At this time, the humidifying chamber 40 communicates with the external environment through the first valve component 51, and the humidifying chamber 40 directly connects to the accommodation space 111 of the water tank 1 through the second valve component 52 and the gas flow channel 41. Further, since both the first opening 305 and the third opening 307 are connected to the humidifying chamber 40 of the humidifying cup 4, external air from the external environment can enter into the humidifying chamber 40 of the humidifying cup 4 through the first valve component 51 and the first opening 305 (as shown by the arrows in FIG. 6A), and the external air in the humidifying chamber 40 can flow to the second valve component 52 through the third opening 307, then pass through the second opening 306, the gas flow channel 41 and enter into the accommodation space 111 of the water tank 1 (as shown by the arrows in FIG. 6B). In this specific embodiment, when the electrolysis module 2 stops operating, the accommodation space 111, the gas flow channel 41, the second valve component 52, the humidifying chamber 40, and first valve component 51 form a continuous channel for external air to enter.

In practice, when the electrolysis module 2 of the hydrogen generator with pressure relief function E operates, the working temperature of the electrolysis module 2 gradually rises, and the temperature of the water tank 1 also increases accordingly. Furthermore, when the electrolysis module 2 stops operating, the working temperature of the electrolysis module 2 gradually decreases, causing a decrease in gas pressure inside the water tank 1, which will result in negative pressure. At this time, the supplementary water in the humidifying cup 4 will enter into the refining device 43 through the micro-bubble structure and then flow into the integrated channel device 3. Next, the supplementary water flows through the condensation flow channel 61 and enters into the condensation filter device 6. Finally, the supplementary water flows from the condensation filter device 6 through the connecting chamber 42 into the water tank 1. Additionally, when the supplementary water flows through the condensation filter device 6, the supplementary water will flush the electrolyte filtered by the condensation filter device 6 back into the water tank 1.

However, due to the hole size of the micro-bubble structure of the refining device 43 are extremely small, and the volume of supplementary water or gas in the humidifying cup 4 after passing through the micro-bubble structure of the refining device 43 is also small, and the hole size of the micro-bubble structure of the refining device 43 is much smaller than the size of the water tank 1. Therefore, when the internal pressure value in the water tank 1 is negative pressure due to the decrease in working temperature of the electrolysis module 2, there is only a portion of the negative pressure can flush the supplementary water in the humidifying cup 4 back into the water tank 1 through the refining device 43. The remaining negative pressure may cause deformation or rupture of the water tank due to the inability to be immediately released and balanced; or the negative pressure inside the water tank 1 may continuously flush the supplementary water in the humidifying cup 4 back into the water tank 1, causing the water level in the water tank 1 to become too high and resulting in excessive pressure and rupture; or the negative pressure inside the water tank 1 may cause the drinking water in the hydrogen water cup 8 to flow back into the water tank 1 through the gas input channel 301 and the refining device 43, thereby contaminating the electrolytic water.

The hydrogen generator device E of the present invention can solve the aforementioned problems. When the electrolysis module 2 stops operating, the first valve component 51 and the second valve component 52 are opened. At this time, the gas in the humidifying chamber 40 will flow into the water tank 1 through the second valve component 52 and the gas flow channel 41 due to the negative pressure in the water tank 1; the external air can flow into the humidifying chamber of the humidifying cup through the first valve component 51, thereby balancing the pressure inside the water tank 1 to the external environment. Therefore, the hydrogen generator device E of the present invention not only prevents deformation or rupture of the water tank 1 due to unbalanced pressure but also avoids continuous flushing the supplementary water from the humidifying cup 4 into the water tank 1, so as to prevent the water level in the water tank 1 from becoming too high. Furthermore, the hydrogen generator device E of the present invention also prevents the drinking water in the hydrogen water cup 8 flow back to the water tank 1, thereby enhancing safety and electrolysis efficiency.

In practice, when the electrolysis module 2 stops operating, the first valve component 51 and the second valve component 52 can be opened after a certain interval. During the certain interval, the supplementary water in the humidifying cup 4 can flush the electrolyte from the condensation flow channel 61 back into the water tank 1 through the refining device 43 due to the negative pressure in the water tank 1.

The positioning of the first valve component and the second valve component can be adjusted from the specific embodiments described above to other configurations. In one specific embodiment, the first valve component is arranged on the humidifying cup, and the second valve component is arranged in the gas flow channel. In practice, the water level of the supplementary water in the humidifying cup can occupy 80% to 90% of the height of the humidifying chamber. The first valve component can be arranged at the upper edge of the outer wall of the humidifying cup to communicate with both the humidifying chamber and the external environment. Furthermore, the other port of the gas flow channel corresponding to the accommodation space of the water tank can directly connect to the upper edge position of the humidifying cup, with the second valve component arranged in the gas flow channel. When the electrolysis module 2 stops operating, the first valve component and the second valve component are opened. At this time, the external air can directly flow into the humidifying chamber, and then flow into the accommodation space of the water tank through the gas flow channel and the second valve component. In addition, the second valve component can also be arranged above the water tank to connect the accommodation space of the water tank and the gas flow channel.

In one embodiment, the hydrogen generator with pressure relief function E further includes a filtering device (the figure is not shown) arranged at the air outlet of the hydrogen generator with pressure relief function E to filter out microbes in the gas comprising hydrogen at the air outlet or to kill bacteria in the gas comprising hydrogen. The components in the filter device can include at least one of activated carbon, nanosilver sputtering, polyethylene terephthalate (PET), and polypropylene (PP) fiber cloth. The antibacterial types can include Staphylococcus aureus, Escherichia coli, Pseudomonas aeruginosa and Methicillin-resistant Staphylococcus aureus, but is not limited thereto. In practice, the air outlet 71 can be the outlet of the nebulizer 7. When the gas comprising hydrogen mixes with the atomized gas to form the health caring gas, the filtering device can first filter out the bacteria in the gas comprising hydrogen flowing through the air outlet 71 before allowing users to inhale it. In one embodiment, the air outlet can be an air outlet pipe to provide the gas comprising hydrogen or the mixed gas, the filtering device can first filter out bacteria in the gas comprising hydrogen or in the mixed gas through the air outlet pipe before allowing users to inhale. In another specific embodiment, the air outlet can also be the outlet of the air outlet pipe connected to the first valve component. When the first valve component is opened, the filtering device will first filter out bacteria in the external air from the air outlet pipe, and then the filtered external air will flow into the humidifying chamber of the humidifying cup through the first valve component. It should be understood that those skilled in the art can adjust the order or the position of the filtering device according to their needs.

The aforementioned filtering device can be different forms and can be arranged differently in the hydrogen generator E or even be arranged outside the hydrogen generator E. In one specific embodiment, the hydrogen generator E can includes an ozone generator coupled to the gas flow channel formed between the integrated channel device, the electrolysis module, and the hydrogen water cup to allow the gas comprising hydrogen flow therein. When the electrolysis module stops operating, the ozone generator can generate ozone into the gas flow channel to sterilize and disinfect. Thus, when the electrolysis module restarts, the gas flow channel has already be cleaned, and there is only few bacteria or no bacteria inside, so as to ensure that the gas comprising hydrogen generated by the electrolysis module is not contaminated by bacteria in the gas flow channel. Furthermore, the ozone generator can also be coupled to the liquid flow channel to provide ozone to sterilize and disinfect in the liquid flow channel.

The ozone generator not only can be arranged inside the hydrogen generator E, but also can be arranged outside the hydrogen generator E. In practice, the hydrogen generator E can have a case to accommodate the units mentioned above, and the ozone generator can be coupled to the gas flow channel formed between the integrated channel device, the electrolysis module, and the hydrogen water cup within the hydrogen generator E to allow the gas comprising hydrogen flow therein, or the ozone generator can be coupled to the liquid flow channel. Therefore, when the electrolysis module 2 stops operating, the ozone generator located outside the hydrogen generator E can generate ozone, which can flow through the gas flow channel or the liquid flow channel in the hydrogen generator E to sterilize and disinfect inside.

In one embodiment, the hydrogen generator E with pressure relief function can further include an ultraviolet (UV) light device (the figure is not shown) arranged in the hydrogen generator E to generate UV light to eliminate microorganisms in the gas comprising hydrogen or to kill bacteria. The ultraviolet light device can generate short-wave ultraviolet light (UV-C) with wavelengths ranging from 100 nm to 280 nm to sterilize the gas. In practice, the ultraviolet light device can be installed in the nebulizer 7 to generate UV light to sterilize the health caring gas, but not limited hereto. The ultraviolet light device can also be arranged in the water tank 1, the integrated channel device 3, the humidifying cup 4, or any pipe through which the gas comprising hydrogen flows therein, to generate UV light to sterilize the above-mentioned components and devices. In addition, the ultraviolet light device can also arranged in the continuous channel. When the external air from the external environment flows into the hydrogen generator E, the ultraviolet light device can generate UV light to sterilize the external air in the continuous channel. In practice, the ultraviolet light device can be arranged at any position in the gas or water flow channel in the hydrogen generator E with pressure relief function to sterilize the gas or water from the gas or water flow channel. Therefore, the hydrogen generator with pressure relief function of the present invention not only can use the alkaline electrolytic water in the water tank and the operating temperature of the electrolysis module to achieve sterilization, but also can use the filtering device or a UV light source to improve sterilization effectiveness and safety.

In one specific embodiment, the hydrogen generator E with pressure relief function further includes a flame arrester 9 coupled to the first valve component 51. The flame arrester 9 can include at least one of a metal mesh filter element and a corrugated filter element to prevent the violent flame of deflagration and withstand the corresponding thermal and mechanical effects. The flame arrester 9 is configured to block off the fire from flowing to the flame arrestor 94, thereby isolating the two spaces to avoid the fire spreading from one side of the flame arrester to the other side, which can cause the fire to spread through the gas flow channel and make an explosion. In this specific embodiment, the flame arrester 9 is arranged on the air outlet pipe 55 connected to the first valve component 51.

In practice, when the electrolysis module 2 operates, the gas comprising hydrogen generated from electrolytic water in the water tank 1 will not only flows through the connecting chamber 42 but also flows through the gas flow channel 41. Additionally, some of the gas comprising hydrogen will be filtered and humidified in the supplementary water in the humidifying chamber 40 by the refining device 43, some of the gas comprising hydrogen will also remain in the space of the humidifying chamber 40. In other words, the continuous channel also contains the gas comprising hydrogen. Furthermore, when the electrolysis module 2 stops operating and the first valve component 51 and the second valve component 52 are opened, the external air can enter into the hydrogen generator E through the first valve component 51 and the gas comprising hydrogen can also flow to the external environment through the first valve component 51, the second valve component 52, and the air outlet pipe 55. Moreover, since the gas comprising hydrogen contains combustible gas components, the flame arrester 9 can prevent the spread of ignited gas from the continuous channel to the exterior of the hydrogen generator E with pressure relief function, thereby enhancing safety. It should be understood that those skilled in the art can add multiple flame arresters and adjust their positions as needed to achieve more interval-based and multi-stage flame suppression, and are not limited hereto.

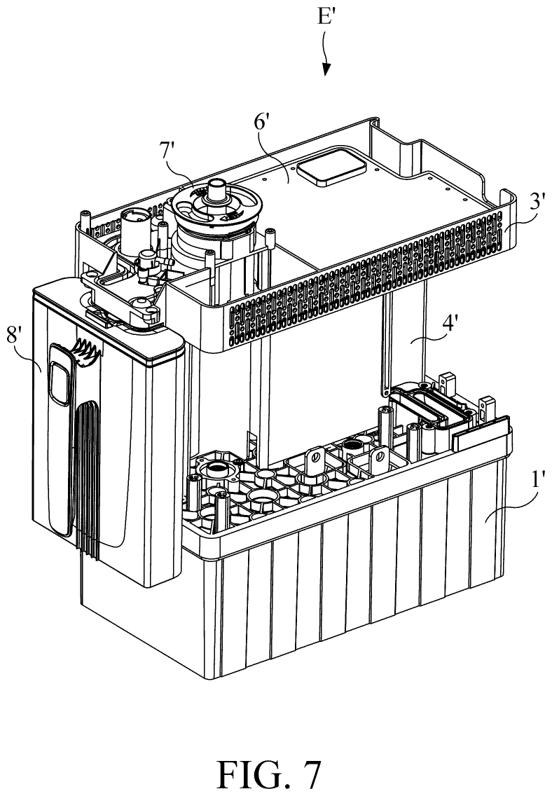

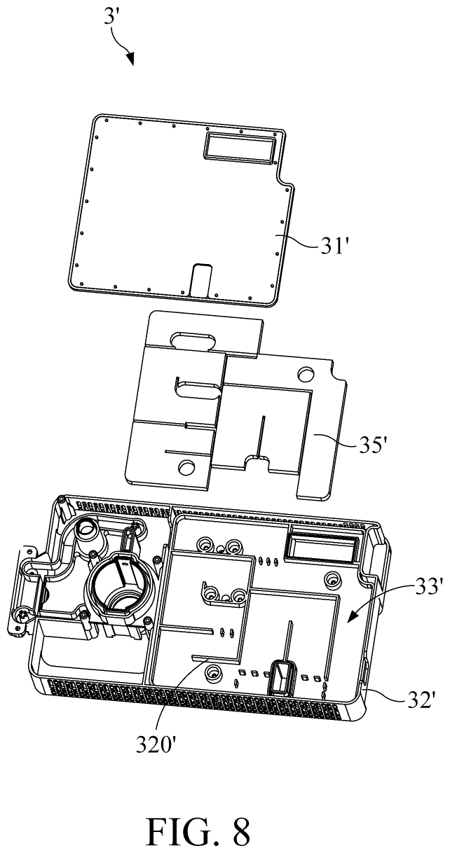

In addition, the condensation filter device of the hydrogen generator with pressure relief function of the present invention can also have other configurations. Please refer to FIG. 7 to FIG. 8. FIG. 7 is a structural schematic diagram illustrating the hydrogen generator E′ with pressure relief function according to another embodiment of the present invention. FIG. 8 is an exploded diagram illustrating the integrated channel device 3′ of FIG. 7. The difference between this specific embodiment and the previous embodiment is that the condensation filter device 6′ of this specific embodiment is fixed in the integrated channel device 3′. As shown in FIG. 7 to FIG. 8, the integrated channel device 3′ includes an upper cover 31′ and a lower cover 32′ having a plurality of specifically arranged spacer plates 320′. When the upper cover 31′ and the lower cover 32′ are coupled together to form a condensation flow channel 33′. Furthermore, the hydrogen generator E′ can have a plurality of filter cottons 35′ arranged in the condensation flow channel 33′ to preliminarily filter impurities in the gas comprising hydrogen. Wherein, the spacer plates 320′ are used to separate the plurality of filter cottons 35′ to prevent from overlapping or contacting each other, which may reduce the effectiveness of condensation moisture absorption. It should be noted that water tank 1′, the humidifying cup 4′, the nebulizer 7′, and the hydrogen water cup 8′ in FIG. 7 are generally similar to those of the corresponding components in the specific embodiments described above and are not further described here.

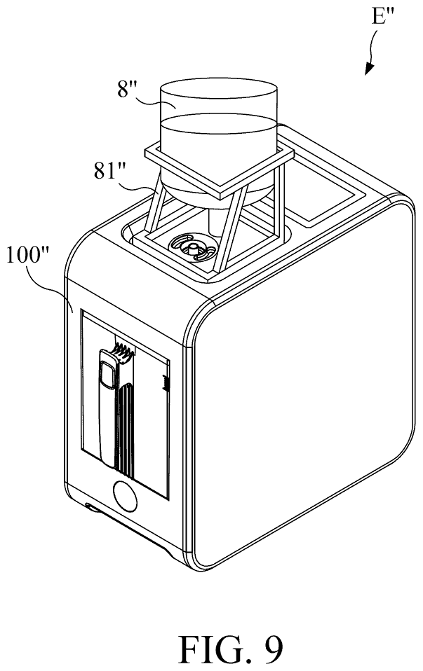

Please refer to FIG. 9. FIG. 9 is a schematic diagram illustrating a hydrogen generator E″ with pressure relief function according to another embodiment of the present invention. In this specific embodiment, the hydrogen generator E″ with pressure relief function further includes a water replenishment port (the figure is not shown) arranged on the integrated channel device and connected to the condensation flow channel of the condensation filter device. In practice, since the long time reaction while the hydrogen generator E″ operating, the water level in the water tank gradually decreases. Users can replenish the water in the water tank through the water replenishment port. When users replenish water through the water replenishment port, the water will flow through the condensation flow channel of the condensation filter device, the connecting chamber, and into the water tank. Additionally, the electrolytes remaining in the condensation flow channel can also be flushed back into the water tank along with the water.

As shown in FIG. 9, the water replenishment port of the hydrogen generator E″ with pressure relief function can also directly connect to a water bracket 8″, and the hydrogen generator E″ with pressure relief function can replenish water in the water tank directly through the supplementary water from the water bucket 8″. In this specific embodiment, the hydrogen generator E″ with pressure relief function further includes a case 100″ for accommodating the water tank, the electrolysis module, the integrated channel device, the condensation filter device, the humidifying cup, the hydrogen water cup, and the nebulizer. The water bucket 8″ is arranged outside the case 100″ and is used to provide supplementary water to flow through the condensation flow channel of the condensation filter device into the water tank. In practice, the hydrogen generator E″ with pressure relief function can further include a controller and control valves (the figure is not shown), and the controller coupled to the control valves. The control valves can be arranged at the water replenishment port, and the controller can control the control valves to open or close the water replenishment port. Additionally, the hydrogen generator E″ with pressure relief function can further include a bracket 81″ arranged on the case 100″ to support the water bucket 8″, thereby increasing stability and safety.

In summary, the hydrogen generator with pressure relief function of the present invention can balance the pressure inside the water tank by controlling the first valve component and the second valve component to be opened to introduce external air to flow therein after the electrolysis module operating, thereby avoiding deformation of the water tank due to pressure differentials and enhancing safety. Moreover, the hydrogen generator with pressure relief function of the present invention can avoid liquids flowing back into the water tank by controlling the first valve component and the second valve component to be opened to block liquids, so as to prevent contamination in the electrolytic water in the water tank or the risk of the water tank bursting due to excessive water levels, thereby improving electrolysis efficiency and safety. Furthermore, due to the alkaline electrolysis environment and the temperature conditions of the hydrogen generator with pressure relief function of the present invention, the environment is unfavorable for bacteria survival, and due to the different devices such as filtering devices, ultraviolet light devices and ozone generators can continuously undergo self-disinfection and self-sterilization to not only provide users with bacteria-free, pure gas comprising hydrogen or health caring gas but also significantly reducing the maintenance costs of the hydrogen generator. Additionally, the hydrogen generator with pressure relief function of the present invention can maintain electrolysis efficiency by backwashing electrolytes with an external water bucket and maintaining the electrolyte concentration in the electrolytic water through the gas baffle assembly to prevent electrolytes from flowing into the humidifying chamber. Moreover, by installing a flame arrester on the continuous channel, the hydrogen generator with pressure relief function of the present invention can prevent the spread of accidentally ignited gas to the outside of the hydrogen generator, thereby enhancing safety.

With the examples and explanations mentioned above, the features and spirits of the invention are hopefully well described. More importantly, the present invention is not limited to the embodiment described herein. Those skilled in the art will readily observe that numerous modifications and alterations of the device may be made while retaining the teachings of the invention. Accordingly, the above disclosure should be construed as limited only by the metes and bounds of the appended claims.

What is claimed is:

1. A hydrogen generator with pressure relief function, comprising:
a water tank having an accommodation space for accommodating an electrolytic water;
an electrolysis module configured in the accommodation space of the water tank, the electrolysis module being configured to receive and electrolyze the electrolytic water from the water tank to generate and output a gas comprising hydrogen;
a humidifying cup configured above the water tank, the humidifying cup having a humidifying chamber and a gas flow channel, the humidifying cup being configured to receive and humidify the gas comprising hydrogen, the humidifying chamber and the gas flow channel being isolated from each other, and the gas flow channel being connected with the water tank;
a first valve component configured to selectively connect the humidifying chamber and an external environment; and
a second valve component configured to selectively connect the accommodation space and the humidifying chamber;
wherein, when the electrolysis module stops operating, an external air from the external environment enters into the accommodation space through the first valve component and the second valve component.

2. The hydrogen generator with pressure relief function of claim 1, wherein when the electrolysis module is operating, the first valve component and the second valve component are closed; when the electrolysis module stops operating, the first valve component and the second valve component are opened, and the accommodation space, the gas flow channel, the first valve component and the second valve component form a continuous channel for allowing the external air to enter the water tank to balance the internal pressure of the water tank.

3. The hydrogen generator with pressure relief function of claim 1, further comprising an integrated channel device configured above the water tank, the integrated channel device comprises a gas input channel for receiving the gas comprising hydrogen and a gas output channel for outputting the gas comprising hydrogen, wherein the first valve component and the second valve component are configured in the integrated channel device.

4. The hydrogen generator with pressure relief function of claim 3, further comprising a nebulizer coupled to the gas output channel for receiving the gas comprising hydrogen, wherein the nebulizer selectively generates an atomized gas to be mixed with the gas comprising hydrogen to form a healthy gas.

5. The hydrogen generator with pressure relief function of claim 3, further comprising a condensation filter device coupled to the integrated channel device, wherein the humidifying cup comprises a connecting chamber for communicating the water tank and the integrated channel device, and the condensation filter device is configured to receive and filter the gas comprising hydrogen from the connecting chamber.

6. The hydrogen generator with pressure relief function of claim 5, further comprising a refining device configured in the humidifying chamber of the humidifying cup and coupled with the condensation filter device, the refining device is configured to refine the gas comprising hydrogen filtered by the condensation filter device, so as to be evenly distribute the gas comprising hydrogen in the humidifying chamber.

7. The hydrogen generator with pressure relief function of claim 3, further comprising a hydrogen water cup for accommodating a liquid, wherein the hydrogen water cup is configured to input the gas comprising hydrogen into the liquid to generate a liquid comprising hydrogen.

8. The hydrogen generator with pressure relief function of claim 3, further comprising an ozone generator, wherein the ozone generator is coupled to a gas flow channel which is formed between the integrated channel device and the electrolysis module for the gas comprising hydrogen to flow therein, wherein the ozone generator is configured to generate an ozone gas into the gas flow channel to sterilize the gas flow channel when the electrolysis module stops electrolyzing.

9. The hydrogen generator with pressure relief function of claim 3, further comprising a germ filtering device configured at an air outlet of the hydrogen generator for filtering out germs from the gas comprising hydrogen at the air outlet.

10. The hydrogen generator with pressure relief function of claim 1, wherein the pH value of the electrolytic water in the electrolysis module is in a range between 13˜13.9.

11. A hydrogen generator with pressure relief function, comprising:
a water tank having an accommodation space for accommodating an electrolytic water;
an electrolysis module configured in the accommodation space of the water tank, the electrolysis module being configured to receive and electrolyze the electrolytic water from the water tank to generate and output a gas comprising hydrogen;
an integrated channel device configured above the water tank, the integrated channel device comprising a gas input channel for receiving the gas comprising hydrogen and a gas output channel for outputting the gas comprising hydrogen;
a pressure relief module being configured to operate to make an external air from the external environment enter into the water tank when the electrolysis module stopping operating; and
a humidifying cup coupling with the integrated channel device for receiving and humidifying the gas comprising hydrogen;
wherein, the pH value of the electrolytic water in the electrolysis module is in a range between 12˜14.

12. The hydrogen generator with pressure relief function of claim 11, further comprising a nebulizer coupled to the gas output channel for receiving the gas comprising hydrogen, wherein the nebulizer selectively generates an atomized gas to be mixed with the gas comprising hydrogen to form a healthy gas.

13. The hydrogen generator with pressure relief function of claim 11, further comprising a germ filtering device configured at an air outlet of the hydrogen generator for filtering out germs from the gas comprising hydrogen at the air outlet.

14. The hydrogen generator with pressure relief function of claim 11, further comprising an ultraviolet light generator configured in the hydrogen generator for generating an ultraviolet light to sterilize the hydrogen generator.

15. The hydrogen generator with pressure relief function of claim 11, further comprising a condensation filter device coupled to the integrated channel device, wherein the humidifying cup comprises a connecting chamber for communicating the water tank and the integrated channel device, and the condensation filter device is configured to receive and filter the gas comprising hydrogen from the connecting chamber.

16. The hydrogen generator with pressure relief function of claim 11, further comprising a hydrogen water cup for accommodating a liquid, wherein the hydrogen water cup is configured to input the gas comprising hydrogen into the liquid to generate a liquid comprising hydrogen.

17. The hydrogen generator with pressure relief function of claim 16, further comprising an ozone generator, wherein the ozone generator is coupled with a gas flow channel which is formed between the integrated channel device, the electrolysis module and the hydrogen water cup for the gas comprising hydrogen to flow therein, wherein the ozone generator is configured to generate an ozone gas into the gas flow channel to sterilize the gas flow channel when the electrolysis module stops electrolyzing.

18. The hydrogen generator with pressure relief function of claim 11, wherein the pressure relief module further comprises a first valve component and a second valve element, the first valve component is configured to selectively connect with the humidifying chamber and the external environment; the second valve element is configured to selectively connect with the accommodation space and the humidifying chamber, when the electrolysis module stops operating, the first valve component and the second valve element are opened, an external air from the external environment enters into the accommodation space through the first valve component and the second valve component.

19. The hydrogen generator with pressure relief function of claim 18, further comprising a flame arrester coupled with the first valve component.

20. The hydrogen generator with pressure relief function of claim 15, further comprising:
a case configured to accommodate the water tank, the electrolysis module, the integrated channel device, the condensation filter device, the pressure relief module and the humidifying cup, and
a bracket configured to support a water bucket, the bracket being arranged outside of the case, the water bucket being configured provide a supplementary water for flowing through the condensation filter device into the water tank.

21. The hydrogen generator with pressure relief function of claim 15, further comprising a gas baffle device configured in the connecting chamber for reducing or avoiding moisture from the gas comprising hydrogen and electrolyte flowing into the integrated channel device.
